(19) United States
(12) Patent Application Publication
(10) Pub. No.: US 2023/0398167 A1
(43) Pub. Date: Dec. 14, 2023

(54) COMPOSITION FOR EXTERMINATING FISH PARASITES

(71) Applicant: Republic of Korea(National Fisheries Research and Development Institute), Busan (KR)

(72) Inventors: Na Young KIM, Busan (KR); Myoung Sug KIM, Busan (KR); Hye Sung CHOI, Busan (KR); Hyun-Ja HAN, Busan (KR); Soo Ji WOO, Busan (KR); Eun Ji JEON, Busan (KR)

(21) Appl. No.: 18/327,798

(22) Filed: Jun. 1, 2023

(30) Foreign Application Priority Data
Jun. 13, 2022 (KR) 10-2022-0071211

(51) Int. Cl. A61K 36/52; A61P 33/00

(52) U.S. Cl. CPC A61K 36/52; A61P 33/00; A61K 2236/33

(57) ABSTRACT

The present invention relates to a composition for exterminating fish parasites comprising an extract of Juglans mandshurica bark as an active ingredient, making it possible to safely and effectively exterminate scuticociliates using an extract derived from a natural material, instead of formalin that is highly harmful, which has been conventionally used to treat scuticociliatosis in fish.

### CROSS-REFERENCE TO RELATED APPLICATION

The priority under 35 USC § 119 of Korean Patent Application 10-2022-0071211 filed Jun. 13, 2022 is hereby claimed, and the disclosure thereof is hereby incorporated herein by reference, in its entirety, for all purposes.

### TECHNICAL FIELD

The present invention relates to a composition for exterminating fish parasites comprising an herbal extract as an active ingredient, and more particularly to a composition for exterminating fish parasites comprising an extract of Juglans mandshurica bark as an active ingredient.

### BACKGROUND ART

Although aquaculture production in Korea is increasing every year, diseases of aquatic organisms have become one of the major issues facing the aquaculture industry due to dense and aging fish farms and coastal pollution. In particular, it has been confirmed that the production of olive flounder (Paralichthys olivaceus), the representative aquaculture fish species, accounts for 50% of the total production, with 43,800 tons out of a total domestic fish farming production of 88,200 tons in 2020 (Statistics Korea, 2021), but that 15.1% of the annual production dies due to diseases such as scuticociliatosis, emaciation disease, streptococcosis, and the others (National Institute of Fisheries Science, 2020).

Since scuticociliatosis was first identified in farmed olive flounder in Jeju in 1990, it has caused mass mortality every year in olive flounder farms across the country due to infection mainly of fry. Recently, serious economic damage is incurred regardless of the size of olive flounder, reaching for 46-57% in cumulative mortality (National Institute of Fisheries Science, 2016). Scuticociliates are ciliophora parasites that cause scuticociliatosis, and there are currently about 20 species, among Miamiensis avidus, which has strong pathogenicity to olive flounder, has been found to be the dominant species (Song et al., Dis. Aquat. Org., 83, 133, 2009). M. avidus is known to have serotypes I and II, and it is assumed that each types show different characteristics.

Currently, there are two active ingredients of formalin and hydrogen peroxide (approved in 2015) for aquatic anthelmintic agent approved for treatment of scuticociliatosis in Korea. Especially, aquatic formalin is most often used anthelmintic agent after first approval in 2006. However, aquatic formalin is approved as a bath agent, and in the case in which scuticociliates infect the brain starting with initial body surface infection and thus cause mass mortality, it is difficult to exterminate the same through bath treatment. Accordingly, questions about the efficacy of aquatic formalin have been raised.

The International Agency for Research on Cancer (IARC) under the World Health Organization (WHO) defines formalin (formaldehyde: CAS No. 50-00-0) as a carcinogen (Group 1) when inhaled or directly exposed to the human body. Based on the approval of aquatic formalin under the premise that there is the safety of human body and aquatic food at the concentrations of formalin (37% formaldehyde) used for treatment of aquatic diseases. In addition, U.S. Food and Drug Administration (FDA) has approved formalin as an aquatic anthelmintic agent after investigation of safety and effectiveness, but the press and environmental organizations are strongly regulated aquatic formalin as a carcinogen and an environmentally destructive substance.

There are mostly research papers and patented technologies for vaccines, chemical preparations, and natural preparations, and thus vaccines are in the pre-commercialization stage, but there are no products for chemical preparations or natural preparations. Due to many side effects caused by chemical agents used at home and abroad, thorough research using natural materials is currently in progress. In particular, regulations on chemical agents will be further strengthened from 2024 due to the introduction of the Positive List System (PLS).

To solve these problems, therefore, the inventors have made great efforts to develop formalin substitutes capable of treating scuticociliate infection effectively using natural materials that are free from residual problems and ascertained that a Juglans mandshurica bark extract is very effective at killing scuticociliates, thus culminating in the present invention.

### SUMMARY OF THE INVENTION

It is an object of the present invention to provide a composition for exterminating fish parasites comprising a safe and highly effective natural extract as an active ingredient.

It is another object of the present invention to provide a method of exterminating fish parasites using the composition described above.

In order to accomplish the above objects, the present invention provides a composition for exterminating fish parasites comprising a Juglans mandshurica bark extract as an active ingredient.

In addition, the present invention provides a method of exterminating fish parasites comprising orally administrating or bath administrating the composition described above.

### BRIEF DESCRIPTION OF DRAWINGS

FIG. 1 shows herbal materials used for screening aquatic anthelmintic agent.

FIG. 2 shows results confirming the cytotoxicity of Juglans mandshurica bark extracts depending on the concentration of ethanol that is used for extraction.

FIG. 3 shows the mortality rates (A: LC₅₀, B: LD₅₀) after administration of the Juglans mandshurica bark extract to olive flounder.

FIG. 4 shows results of blood biochemical analysis of an acute toxicity test (LC₅₀) after bath administration of olive flounder with the Juglans mandshurica bark extract (ALP, alanine aminotransferase; GOT, glutamic oxalacetic transaminase; GPT, glutamic pyruvate transaminase; GLU, glucose; BUN, blood urea nitrogen).

FIG. 5 shows scuticociliate extermination effects depending on the type of administration of the Juglans mandshurica bark extract to olive flounder.

### DETAILED DESCRIPTION AND PREFERRED EMBODIMENTS OF THE INVENTION

Unless otherwise defined, all technical and scientific terms used herein have the same meanings as those typically understood by those skilled in the art to which the present invention belongs. In general, the nomenclature used herein is well known in the art and is typical.

In the present invention, in order to exterminate scuticociliates, which are fish parasites causing mass mortality in aquaculture olive flounder, using a natural extract that is free from residual problems and is safe and highly efficacious, herbal extracts are prepared, and extracts having scuticociliate killing activity are screened, confirming that the ethanol extract of Juglans mandshurica bark exhibits high scuticociliate killing activity.

Accordingly, in an aspect, the present invention relates to a composition for exterminating fish parasites comprising an extract of Juglans mandshurica bark as an active ingredient.

In the present invention, the fish parasite may be a scuticociliate.

In the present invention, the extract of Juglans mandshurica bark may be an extract using a solvent selected from the group consisting of ethanol, methanol, chloroform, and water, and is preferably an ethanol extract.

The ethanol extract of Juglans mandshurica bark according to the present invention may be a 100-10% ethanol extract, preferably a 90-20% ethanol extract, more preferably an 80-30% ethanol extract, much more preferably a 70-40% ethanol extract, even much more preferably a 60-45% ethanol extract.

In the present invention, the Juglans mandshurica bark is the bark of Juglans mandshurica, and there are 15 species of ‘Juglandaceae’ and ‘Juglans’ worldwide, and 4 species including those cultivated in Korea.

The four species cultivated in Korea are Juglans mandshurica (Juglans cathayensis Dode, Juglans draconis Dode, Juglans formosana Hayata), Juglans regia (walnut tree, Persian walnut), Juglans cordiformis, and Juglans sieboldiana (Juglans mandshurica var. sieboldiana Makino). Juglans mandshurica contains large amounts of hydrojuglone and tannins, along with quinone, phenol, flavonoids, coumarine, lignan, and steroids. It is known that the bark, root, leaf, unripe fruit peel, etc. of Juglans mandshurica have anthelmintic agent and sterilization effects, and particularly hydrojuglone secreted from the root has cytotoxic and anti-inflammatory effects, and tannin has disinfecting and anti-inflammatory effects through astringent action.

In another aspect, the present invention relates to a method of exterminating fish parasites comprising orally administrating or bath administrating the composition described above.

In the present invention, the fish parasite may be a scuticociliate.

A better understanding of the present invention may be obtained through the following examples. These examples are merely set forth to illustrate the present invention, and are not to be construed as limiting the scope of the present invention, as will be apparent to those skilled in the art.

### Example 1: Production of Aquatic Anthelmintic Agent Candidates Derived from Natural Materials

Herbal materials and seaweeds were chosen and extracted as candidates for aquatic anthelmintic agent. Herbal extracts were prepared using different solvents (hot water and ethanol). A solvent was added to 50 g of the herbal material, followed by ultrasonic extraction (three times), concentration under reduced pressure, and freeze-drying to eliminate the effect of the extraction solvent. Then, the resulting product was dissolved in dimethyl sulfoxide (DMSO), administered at different concentrations, and used to measure antiparasitic efficacy.

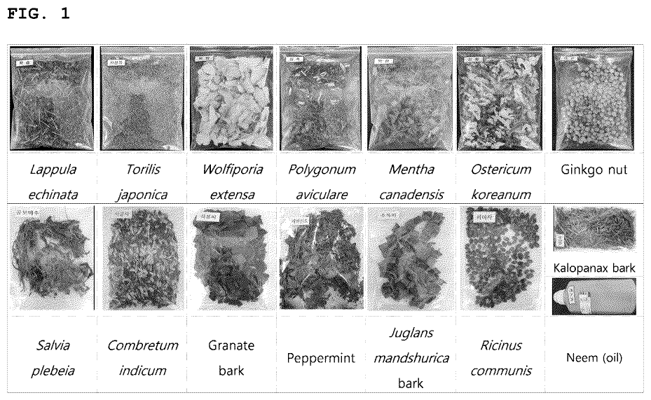

Extracts of 15 herbal materials (FIG. 1) known to have antiparasitic efficacy were prepared using solvents (ethanol and hot water), concentrated under reduced pressure, and freeze-dried, after which the efficacy against scuticociliates depending on the concentration (0 to 10,000 mg/L) thereof was measured. Therefore, antiparasitic efficacy was confirmed in Ostericum koreanum, Juglans mandshurica bark, Torilis japonica, and Lappula echinata, and the criterion for efficacy was determined to be 100 mg/L, similar to the aquatic formalin dosage concentration (Table 1).

### Example 2: Confirmation of Antiparasitic Efficacy of Herbal Materials and Investigation of Drug Effect Concentration

The ability of the herbal extracts prepared in Example 1 to kill scuticociliates was confirmed.

Scuticociliates were isolated from the abdominal cavity of subjects infected therewith, inoculated into Hirame natural embryo (HINAE) cells, and cultured in MEM (minimum essential media, Welgene) supplemented with 1% antibiotics (Gibco) and 10% FBS (Gibco) in an incubator at 20° C. After culture for 3 to 4 days to full confluency, scuticociliates were centrifuged at 3000 g and 4° C. for 10 minutes, and the supernatant was removed to separate scuticociliates, which were then dispensed at 1×10⁴/well into a 96-well plate and used in experiments. The extract was added at different concentrations to the cultured scuticociliates, and the killing efficacy (extent of killing of scuticociliates, movement, and extent of cytoplasmic destruction) was observed at intervals of 10 minutes, 30 minutes, 1 hour, 2 hours, and 4 hours in an incubator at 20° C.

The efficacy of killing scuticociliates was evaluated to be the highest in Ostericum koreanum, but strong fish toxicity was confirmed upon oral administration of Ostericum koreanum and thus histopathologic examination was carried out. Consequently, exfoliation of the gill epithelium, vacuolar degeneration of the kidneys, and liver atrophy were observed, and drug toxicity response appeared generally, and the efficacy was ultimately determined in the order of Juglans mandshurica bark>Lappula echinata>Ostericum koreanum.

TABLE 1
Confirmation of ability of herbal materials to kill
scuticociliates

| | | | | 100% EtOH extract dosage | | | | |
|---|---|---|---|---|---|---|---|---|
| | Treatment | | | concentration (mg/L) | | | | |
| Candidate | time | | Control | 1 | 10 | 100 | 1,000 | 10,000 |
| Ostericum | 10 | min | 100% | 100% | 100% | 80% | Death | Death |
| koreanum | 30 | min | 100% | 100% | 100% | 50% | Death | Death |
| | 1 | h | 100% | 100% | 100% | Death | Death | Death |
| | 2 | h | 100% | 100% | 100% | Death | Death | Death |
| | 4 | h | 100% | 100% | 100% | Death | Death | Death |
| Juglans | 10 | min | 100% | 100% | 100% | 100% | Death | Death |
| mand- | 30 | min | 100% | 100% | 100% | 50% | Death | Death |
| shurica | 1 | h | 100% | 100% | 100% | 10% | Death | Death |
| bark | 2 | h | 100% | 100% | 100% | Death | Death | Death |
| | 4 | h | 100% | 100% | 100% | Death | Death | Death |
| Torilis | 10 | min | 100% | 100% | 100% | 100% | 100% | Death |
| japonica | 30 | min | 100% | 100% | 100% | 100% | 50% | Death |
| | 1 | h | 100% | 100% | 100% | 50% | 50% | Death |
| | 2 | h | 100% | 100% | 100% | 30% | Death | Death |
| | 4 | h | 100% | 100% | 100% | Death | Death | Death |
| Lappula | 10 | min | 100% | 100% | 100% | 100% | Death | Death |
| echinata | 30 | min | 100% | 100% | 100% | 100% | Death | Death |
| | 1 | h | 100% | 100% | 100% | 100% | Death | Death |
| | 2 | h | 100% | 100% | 100% | 50% | Death | Death |
| | 4 | h | 100% | 100% | 100% | 50% | Death | Death |

Thus, the Juglans mandshurica bark extract was chosen as the final candidate, and a 50% ethanol extract was ultimately determined for commercialization of safe aquatic anthelmintic agent according to alcohol methods.

TABLE 2

| | | | | 100% EtOH extract dosage | | | | |
|---|---|---|---|---|---|---|---|---|
| | Treatment | | | concentration (mg/L) | | | | |
| Candidate | time | | Control | 1 | 10 | 100 | 1,000 | 10,000 |
| Juglans | 10 | min | 100% | 100% | 100% | 100% | Death | Death |
| mand- | 30 | min | 100% | 100% | 100% | 70% | Death | Death |
| shurica | 1 | h | 100% | 100% | 100% | 20% | Death | Death |
| bark | 2 | h | 100% | 100% | 100% | Death | Death | Death |
| 100% EtOH | 4 | h | 100% | 100% | 100% | Death | Death | Death |
| Juglans | 10 | min | 100% | 100% | 100% | 20% | Death | Death |
| mand- | 30 | min | 100% | 100% | 100% | 20% | Death | Death |
| shurica | 1 | h | 100% | 100% | 100% | Death | Death | Death |
| bark | 2 | h | 100% | 100% | 100% | Death | Death | Death |
| 70% EtOH | 4 | h | 100% | 100% | 100% | Death | Death | Death |
| Juglans | 10 | min | 100% | 100% | 100% | 20% | Death | Death |
| mand- | 30 | min | 100% | 100% | 100% | 20% | Death | Death |
| shurica | 1 | h | 100% | 100% | 100% | Death | Death | Death |
| bark | 2 | h | 100% | 100% | 100% | Death | Death | Death |
| 50% EtOH | 4 | h | 100% | 100% | 100% | Death | Death | Death |

In addition, experimentation was conducted to determine a specific active concentration of the 50% ethanol extract of Juglans mandshurica bark, confirming that scuticociliates were killed at a final concentration of 40 mg/L. Accordingly, experimentation was performed at 40 mg/L.

TABLE 3
Confirmation of scuticociliate killing activity of 50%
ethanol extract of Juglans mandshurica bark depending on
dosage concentration and treatment time

| | | | 50% EtOH extract dosage | | | | | | |
|---|---|---|---|---|---|---|---|---|---|
| | Treatment | | concentration (mg/ L) | | | | | | |
| Candidate | time | Control | 10 | 20 | 40 | 60 | 80 | 100 | 200 |
| Juglans | 10 min | 100% | 100% | 100% | 100% | 90% | 90% | 60% | 60% |
| mandshurica | 30 min | 100% | 100% | 90% | 60% | 50% | 40% | 50% | 50% |
| bark | 1 h | 100% | 100% | 80% | 50% | 30% | Death | Death | Death |
| 50% EtOH | 2 h | 100% | 100% | 80% | Death | Death | Death | Death | Death |
| | 4 h | 100% | 100% | 80% | Death | Death | Death | Death | Death |

### Example 3: Confirmation of Cytotoxicity of Herbal Extract

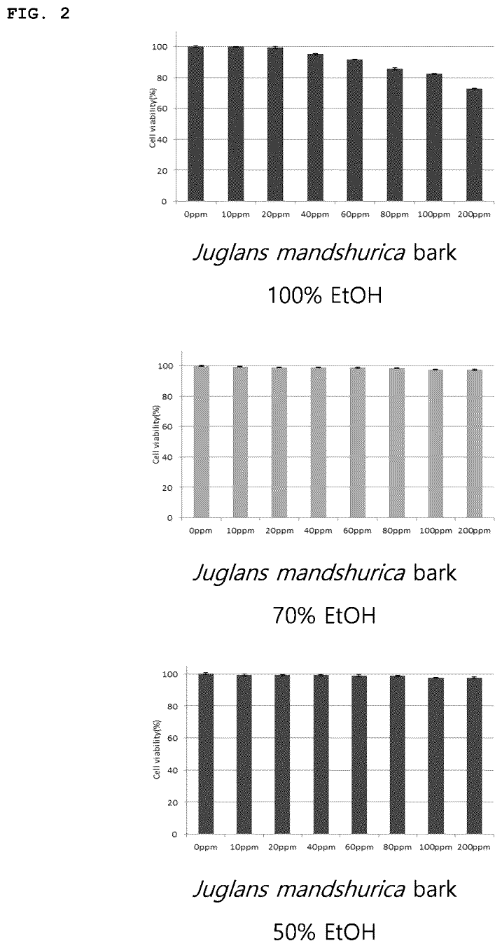

Measurement was performed on cytotoxicity of the prepared extracts using HINAE cells. Cells at 1×10⁵/well were cultured to 80% density at 20° C. for 24 hours, and then treated with the extracts at different concentrations (0 to 1,000 mg/L). After incubation for 48 hours, absorbance was measured at 450 nm using a cytotoxicity measurement drug (CCK-8, Dojindo). Based on results, as shown in FIG. 2, cytotoxicity was exhibited at concentrations of 40 mg/L or more when using the 100% ethanol extract of Juglans mandshurica bark, but did not appear even at a concentration of 200 mg/L when using the 50% ethanol extract of Juglans mandshurica bark.

### Example 4: Fish Acute Toxicity Test of Natural Aquatic Anthelmintic Agent

In order to evaluate the safety of Juglans mandshurica bark, an acute toxicity test (lethal concentration 50%: LC₅₀, lethal dose 50%: LD₅₀) after bath administration or oral administration was conducted in 10 olive flounder (13.9±0.9 cm, 27.1±4.6 g) for each experimental group. A 50% ethanol extract of Juglans mandshurica bark was prepared at 0 to 1,000 mg/L and used up to 1,000 mg/L, including the control, for bath administration, and was prepared at concentrations of up to 400 mg/kg B.W. and used for oral administration. After addition of the extract at each concentration, mortality rates were measured every day for 96 hours, and blood and histopathological examinations of surviving subjects were performed. For histopathological analysis, the gills, heart, intestines, kidneys, liver, and spleen of experimental fish were fixed in 10% neutral formalin, dehydrated, and paraffin-embedded, and the sliced tissues were stained with hematoxylin-eosin (H&E) and observed under a microscope.

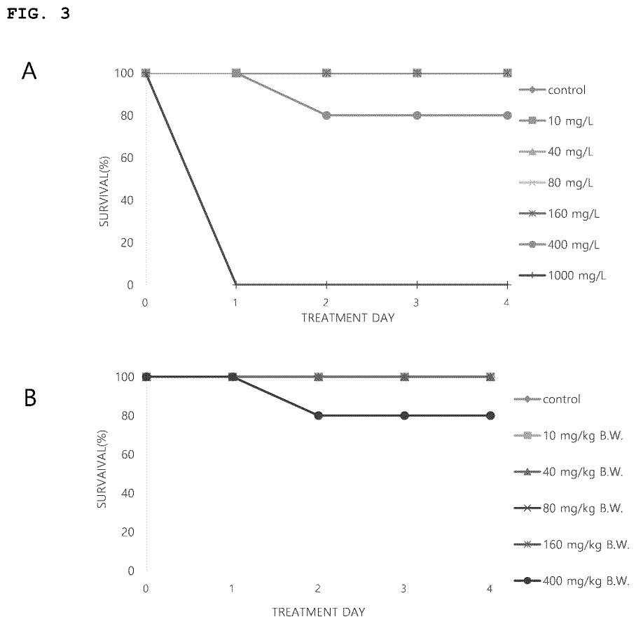

Based on the results, as shown in FIG. 3, LC₅₀ (96 h) was measured to be 509.06 mg/L, and LD₅₀ (96 h) was measured to be 805.8 mg/kg B.W.

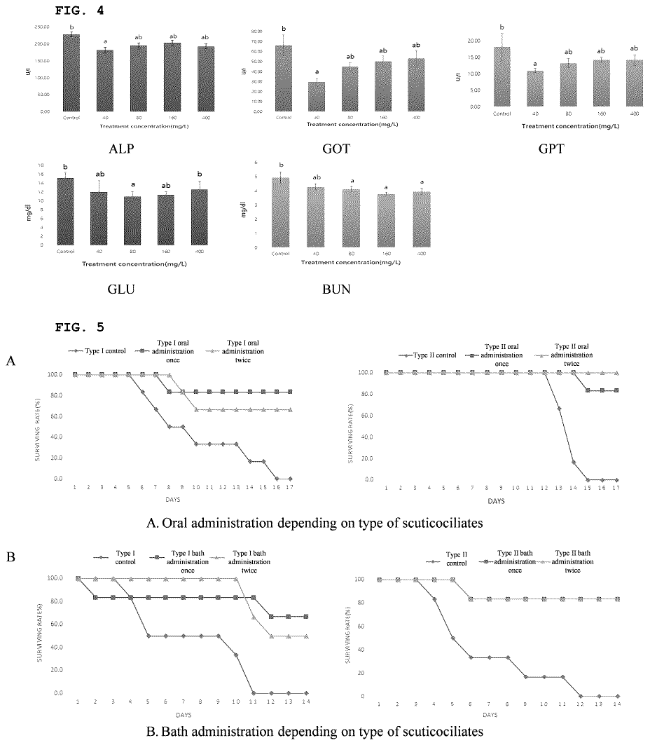

Based on results of blood biochemical analysis of the acute toxicity test (LC₅₀) after bath administration, as shown in FIG. 4, the items that are functional indicators of the liver and kidneys, which are the main target organs for drug toxicity, were not different compared to the control. Thereamong, GOT and GPT levels showed a tendency to greatly decrease at 40 mg/L, which was judged to be closely related to the hepatoprotective effect among the effects of Juglans mandshurica bark.

Based on results of acute toxicity evaluation, in the acute toxicity test (LC₅₀, LD₅₀) after bath or oral administration of olive flounder using the Juglans mandshurica bark extract, this extract did not show any harmful effects on blood biochemistry and histopathology at 160 mg/L and 160 mg/kg B.W., indicating safety.

### Example 5: Confirmation of Efficacy of Juglans mandshurica Bark Extract as Anthelmintic Agent

Healthy olive flounder without disease were purchased from olive flounder farms in Chungcheongnam-do, Korea, and used as experimental fish after acclimatization for 2 weeks at a water temperature of 20±1° C. in a seawater flow-through land tank. On the 2^{nd} day after artificially infecting 10 experimental fish for each group with 1×10⁵ cell/fish of scuticociliates (I, II) stored in the Pathology Research Department of the National Institute of Fisheries Science (Type I and II scuticociliates), the extract was added to feed pellets at a concentration of 40 mg/L, and the fish were anesthetized and subjected to oral administration or bath administration therewith once or twice. To this end, a powdery feed mixture was formed into feed pellets (about 0.25 g), after which a Juglans mandshurica bark powder was added to a concentration of 40 mg/kg B.W., followed by coating with a feed paste once more. In the control for oral administration, feed pellets, prepared in the same manner with the exception that the extract was not added, were orally administered to fish that were anesthetized. Then, fish death by scuticociliates was confirmed.

The results of scuticociliate extermination effect are shown in FIG. 5. There was no significant pathogenicity due to a difference in the type of scuticociliates, and based on results of fish death caused by scuticociliates for 17 days, the oral administration group showed a relative survival rate of 60-80%, and the bath administration group showed a relative survival rate of 50-80%.

### INDUSTRIAL APPLICABILITY

According to the present invention, it is possible to safely and effectively exterminate scuticociliates using an extract derived from a natural material, instead of formalin that is highly harmful, which has been conventionally used to treat scuticociliatosis in fish.

Although specific embodiments of the present invention have been disclosed in detail above, it will be obvious to those skilled in the art that the description is merely of preferable exemplary embodiments and is not to be construed as limiting the scope of the present invention. Therefore, the substantial scope of the present invention will be defined by the appended claims and equivalents. Simple modifications or alterations of the present invention may be easily used by those of ordinary skill in the art, and all such modifications or alterations may be considered to be included in the scope of the present invention.

1. A composition for exterminating a fish parasite comprising an extract of Juglans mandshurica bark as an active ingredient.

2. The composition according to claim 1, wherein the parasite is a scuticociliate.

3. The composition according to claim 1, wherein the extract of Juglans mandshurica bark is an extract using a solvent selected from the group consisting of ethanol, methanol, chloroform, and water.

4. The composition according to claim 1, wherein the extract of Juglans mandshurica bark is an ethanol extract.

5. A method of exterminating a fish parasite comprising orally administrating or bath administrating the composition according to claim 1.

6. The method according to claim 5, wherein the fish parasite is a scuticociliate.
